(19) United States
(12) Patent Application Publication
(10) Pub. No.: US 2010/0204324 A1
(43) Pub. Date: Aug. 12, 2010

(54) Optically Active N-(Alpha-Mercaptopropionyl)Glycine

(75) Inventors: Yong Wang, Nanjing (CN); Cang Zhang, Nanjing (CN); Zaijin Teng, Nanjing (CN); Wenping Zhang, Nanjing (CN); Jinye Qian, Nanjing (CN)

Correspondence Address: THE WEBB LAW FIRM, P.C., 700 KOPPERS BUILDING, 436 SEVENTH AVENUE, PITTSBURGH, PA, 15219, US

(73) Assignee: NANJING SANHOME PHARMACEUTICAL CO., LTD., Nanjing (CN)

(21) Appl. No.: 12/668,588

(22) PCT Filed: Dec. 28, 2007
(86) PCT No.: PCT/CN07/03859

§ 371 Date: Jan. 11, 2010

(30) Foreign Application Priority Data
Jul. 12, 2007 (CN) 200710137428.0

(51) Int. Cl. A61K 31/198; C07C 323/41; A61P 1/16; C12Q 1/02; G01N 30/84

(52) U.S. Cl. 514/554; 562/556; 514/562; 435/29; 73/61.55

(57) ABSTRACT

An optically active N-(α-mercaptopropionyl)glycine, i.e., R-(−)-N-(α-mercaptopropionyl)glycine or S-(−)-N-(α-mercaptopropionyl)glycine, a preparation method thereof, a pharmaceutical preparation containing the compound or a pharmaceutically acceptable salt or ester thereof, and use of the same in preparation of detoxification medicament for improving metabolism, are provided.

### BACKGROUND OF THE INVENTION

1. Field of the Invention

The present invention relates to an optically active N-(α-mercaptopropionyl)glycine, preparation method thereof, use of the same in preparation of detoxification medicament for improving metabolism, and pharmaceutical preparation containing the same.

2. Related Art

N-(α-mercaptopropionyl)glycine, generic name tiopronin, is used to treat, for example, acute and chronic hepatitis, and cirrhosis in clinics, with very excellent therapeutic use in treatment of liver diseases, and preparation method and pharmaceutical preparation thereof have been frequently disclosed. A tiopronin pharmaceutical preparation is disclosed in Chinese Patent Application No. CN02129300.7, and a process for synthesizing tiopronin is disclosed in Chinese Journal of Medicinal Chemistry (Vol. 7, No. 1, p. 55-56, March 1997). However, there is not any report for the optical activity, i.e., levo and dextro isomers of N-(α-mercaptopropionyl)glycine. For the majority of the drugs, a single enantiomer has high therapeutic efficacy and low adverse effect, and thus is called as eutomer, while the inactive or low-active enantiomer is referred to as distomer; and in most cases, the distomer has no therapeutic efficacy, but can also partly neutralize the effect of the enantiomer, and even incur serious adverse effect. The enantiomeric drugs have very different pharmacologic activity and metabolic process in the body, and thus exhibit different biological activity and efficacy. In clinical application, due to the lack of awareness of the differences in pharmacodynamic and pharmacokinetic behaviors of the individual enantiomer of chiral drugs, the conclusions sometimes contradict the therapeutic efficacy or occurrence of adverse effect, and will even improperly direct the application of drugs in clinics. Therefore, researches on optical activities of drugs are very necessary.

### SUMMARY OF THE INVENTION

The present invention is directed to an optically active N-(α-mercaptopropionyl)glycine.

The present invention is further directed to a method for preparing the optically active N-(α-mercaptopropionyl)glycine.

The present invention is further directed to a use of the optically active N-(α-mercaptopropionyl)glycine in preparation of detoxification medicament for improving metabolism.

The present invention is further directed to a pharmaceutical preparation containing the optically active N-(α-mercaptopropionyl)glycine.

As embodied and broadly described herein, the present invention provides an optically active N-(α-mercaptopropionyl)glycine, which is levo or dextro N-(α-mercaptopropionyl)glycine.

The levo N-(α-mercaptopropionyl)glycine is R-(−)-N-(α-mercaptopropionyl)glycine

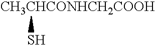

of R-configuration, or a pharmaceutically acceptable salt or ester thereof, in which the salt can be an amino acid salt or a metal salt.

The amino acid salt has a general structural formula below:

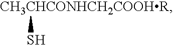

in which R can be an amino acid selected from, for example, arginine, lysine, glycine, aspartic acid, alanine, phenylalanine, leucine, isoleucine, ornithine, cystine, cysteine, tyrosine, valine, serine, histidine, threonine, tryptoophan, methionine, methionine, proline, glutamic acid, and hydroxyproline.

The metal salt has a general structural formula below:

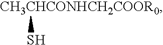

in which R₀ can be, for example, potassium or sodium.

The ester has a general structural formula below:

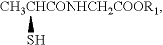

in which R₁ is a linear C₁-C₅ alkyl group.

The dextro N-(α-mercaptopropionyl)glycine is S-(+)-N-(α-mercaptopropionyl)glycine

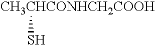

of S-configuration, or a pharmaceutically acceptable salt or ester thereof, in which the salt can be an amino acid salt or a metal salt.

The amino acid salt has a general structural formula below:

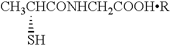

in which R can be an amino acid selected from, for example, arginine, lysine, glycine, aspartic acid, alanine, phenylalanine, leucine, isoleucine, ornithine, cystine, cysteine, tyrosine, valine, serine, histidine, threonine, tryptoophan, methionine, methionine, proline, glutamic acid, and hydroxyproline.

The metal salt has a general structural formula below:

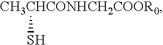

in which R₀ can be, for example, potassium or sodium.

The ester has a general structural formula below:

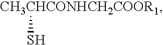

in which R₁ is a linear C₁-C₅ alkyl group.

The present invention also provides a method for preparing the optically active N-(α-mercaptopropionyl)glycine, which includes a method for preparing R-(−)-N-(α-mercaptopropionyl)glycine and a method for preparing S-(−)-N-(α-mercaptopropionyl)glycine.

I. The method for preparing R-(−)-N-(α-mercaptopropionyl)glycine includes:

1. reacting R-(+)-2-chloropropionic acid with thionyl chloride to get R-(+)-2-chloropropionyl chloride;

2. reacting R-(+)-2-chloropropionyl chloride with glycine under a weak basic condition to get R-(+)-2-chloropropionyl glycine; and

3. reacting sodium sulfide with sublimed sulfur to get sodium disulfide, which is then acidified by reacting with R-(+)-2-chloropropionyl glycine, to get R-(−)-N-(α-mercaptopropionyl)glycine;

where the resulting R-(−)-N-(α-mercaptopropionyl)glycine is further reacted with an acid, a base, or an alcohol to get a salt or an ester.

II. The method for preparing S-(+)-N-(α-mercaptopropionyl)glycine includes:

1. reacting S-(−)-2-chloropropionic acid with thionyl chloride to get S-(−)-2-chloropropionyl chloride;

2. reacting S-(−)-2-chloropropionyl chloride with glycine under a weak basic condition to get S-(−)-2-chloropropionyl glycine; and

3. reacting sodium sulfide with sublimed sulfur to get sodium disulfide, which is then acidified by reacting with S-(−)-2-chloropropionyl glycine, to get S-(+)-N-(α-mercaptopropionyl)glycine;

where the resulting S-(+)-N-(α-mercaptopropionyl)glycine is further reacted with an acid, a base, or an alcohol to get a salt or an ester.

The present invention also provides a use of the optically active N-(α-mercaptopropionyl)glycine, i.e., R-(−)-N-(α-mercaptopropionyl)glycine or S-(+)-N-(α-mercaptopropionyl)glycine, or a pharmaceutically acceptable salt or ester thereof in preparation of detoxification medicament for improving metabolism, and particularly a use in treatment of acute and chronic liver diseases and improvement of liver function, including protection of liver tissue cells, treatment of various hepatitis, for example, acute and chronic hepatitis, viral hepatitis, alcoholic hepatitis, drug induced hepatitis, and heavy metal toxic hepatitis, and treatment of fatty liver, acute and chronic liver injury, and cirrhosis; as well as in prevention and cure of peripheral blood leukopenia caused by chemoradiation therapy and accelerating of restoration of liver cells, to reduce the adverse effect of chemotherapy; in prevention and cure of early senile cataract and vitreous opacities; and in heavy metal detoxification.

It is found from pharmacodynamic experiment that both R-(−)-N-(α-mercaptopropionyl)glycine and S-(+)-N-(α-mercaptopropionyl)glycine have good protection effect for liver injury, which is better than that of N-(α-mercaptopropionyl)glycine.

It is found from pharmacokinetic experiment that, conversion of R-(−)-N-(α-mercaptopropionyl)glycine and S-(+)-N-(α-mercaptopropionyl)glycine to each other have not occurred in body. The specific experiment is a pharmacokinetic test in the body of a test animal, including determining the plasma drug concentration with high-performance liquid chromatography-mass spectrometry (HPLC-MS).

The process for treat a plasma sample to formulate an injected solution includes plasma acidification, extraction, and then derivatization, and is as follows:

1. plasma acidification, where the acid used for acidification can be, for example, hydrochloric acid, phosphoric acid, perchloric acid, or acetic acid, with hydrochloric acid being preferred, and hydrochloric acid of 1 mol/L being more preferred; and where the volume ratio of the acid to the plasma sample is (150 μl-250 μl):(2 ml-4 ml), with 200 μl:3 ml being preferred;

2. extraction of the acidified plasma, where the organic solvent used for extraction can be, for example, ethyl acetate, chloroform, trichloromethane, diethyl ether, or n-hexane, with ethyl acetate being preferred; and

3. derivatization of the extract, where the useful derivatization reagent can be, for example, phenyl isothiocyanate and 2,3,4,6-tetra-O-acetyl-β-D-pyranoglucose isothiocyanate (GITC) solution, with GITC solution being preferred, GITC solution of 2 mg/ml being more preferred, and solution of 2 mg/ml GITC in tetrahydrofuran being most preferred; where the derivatization temperature is 15-45° C., with 25-35° C. being preferred, and 30° C. being more preferred; and where the derivatization time is 10-30 min, with 15-25 min being preferred, and 20 min being more preferred.

Chromatographic conditions include:

mobile phase A: methanol; mobile phase B: an aqueous solution, containing 0.05-0.20 mmol/L of sodium chloride and 5.0-6.0 mmol/L of formic acid, with 0.10 mmol/L of sodium chloride and 5.3 mmol/L of formic acid being preferred, where the ratio of A to B is (40-50:50-60, with 44:56 being preferred).

Mass spectrometric conditions include:

ionization mode: electrospray ionization; selective ion detection; curved desolvation line (CDL); temperature: 200° C.-300° C., with 250° C. being preferred; heating block temperature: 150° C.-250° C., with 200° C. being preferred; CDL voltage: 20V-30V, with 25V being preferred; detection voltage: +1.2 kV-+1.8 kV, with +1.50 kV being preferred; flow rate of atomizing gas: 1.2 L/min-1.8 L/min, with 1.5 L/min being preferred; flow rate of drying gas: 1.5 L/min-2.5 L/min, with 2.0 L/min being preferred; detected ion: derivative of test drug [M+Na]+(m/z): 575.20; internal standard: derivative of N-isobutanoyl-D-cysteine (NIDC) [M+Na]+(m/z): 603.05;

as long as the separation degree of the internal standard peak from the major peak meets the requirements of Chinese Pharmacopoeia.

The results indicate that, in spectrogram of R-(−)-N-(α-mercaptopropionyl)glycine at each time points after being administrated individually, the presence of S-(+)-N-(α-mercaptopropionyl)glycine is not obviously detected; likewise, the presence of R-(−)-N-(α-mercaptopropionyl)glycine is not obviously detected either after administrating S-(+)-N-(α-mercaptopropionyl)glycine, which suggest that conversions to each other have not occurred in body.

Therefore, it is practicable to make clinically useful pharmaceutical preparation from R-(−)-N-(α-mercaptopropionyl)glycine or S-(+)-N-(α-mercaptopropionyl)glycine.

In the present invention, the optically active N-(α-mercaptopropionyl)glycine is made into a clinically useful pharmaceutical preparation, which contains R-(−)-N-(α-mercaptopropionyl)glycine or S-(+)-N-(α-mercaptopropionyl)glycine, or a pharmaceutically acceptable salt or ester thereof as active ingredient, and a pharmaceutically acceptable adjuvant, and is an orally administrated preparation or an injection preparation.

The orally administrated preparation includes: a general oral preparation, for example, tablet, capsule, granule, chewable tablet, or effervescent tablet; a rapid release preparation, for example, dispersible tablet, or orally disintegrating tablet; or a slow release preparation, for example, slow release tablet, or slow release pellet. The injection preparation includes, for example, injectable solution, concentrated solution for injection, or sterile powder for injection.

The adjuvant in the oral pharmaceutical preparation includes a filler, a binder, or a disintegrant, in which the weight contents of the filler and the disintegrant are 10-60%, and 2-30% respectively; and a glidant, a lubricant, and a surfactant can optionally exist, in which the weight contents of the glidant, the lubricant, and the surfactant are 0.1-5%, 0.1-5%, and 0.005-1% respectively. The filler can be starch, pregelatinized starch, carboxymethyl starch, microcrystalline cellulose, lactose, dextrin, sucrose, glucose, mannitol, sorbitol, calcium sulfate dihydrate, dibasic calcium phosphate, tribasic calcium phosphate, or calcium carbonate. The binder can be corn starch, pregelatinized corn starch, pregelatinized starch, gelatine, sucrose, arabic gum, povidone, methylcellulose of various viscosities, sodium carboxymethyl cellulose of low viscosity, ethylcellulose of various viscosities, polyvinyl alcohol of various viscosities, polyethylene glycol 6000, or solution of hydroxypropylmethyl cellulose in water or an alcohol. The disintegrant can be starch, pregelatinized starch, low-substituted hydroxypropyl cellulose, microcrystalline cellulose, absolute lignocellulose, alginic acid, sodium carboxymethyl starch, croscarmellose sodium, guar gum, crosslinked polyvinylpyrrolidone, ion exchange resin, methylcellulose, carboxymethylcellulose sodium, and effervescent disintegrant composed of an organic acid (e.g., citric acid, and tartaric acid) and carbonate (e.g., sodium carbonate and sodium bicarbonate). The lubricant can be tartaric acid, magnesium stearate, calcium stearate, zinc stearate, talc, polyethylene glycol 4000, polyethylene glycol 6000, polyethylene glycol 8000, magnesium lauryl sulfate, sodium benzoate, sodium acetate, sodium chloride, sodium oleate, boric acid, leucine, adipic acid, fumaric acid, glycerol triacetate, polyoxyethylene monostearate, monolauryl saccharate, magnesium lauryl sulfate, and sodium lauryl sulfate. The glidant can be gas phase micro-powder silica gel, synthetic micro-powder silica gel, and magnesia. The surfactant can be sodium dodecylsulfate, poloxamer, Tweens, Spans, hexadecyl trimethylamine bromide, sodium lauryl sulfate, sodium stearate sulfonate, polyoxyethylene castor oil, and polyoxyethylene monostearate. The prescription can optionally contain flavor, including stevioside, fructose, sucrose, glucose, aspartame, protein sugar, xylitol, mannitol, sorbitol, lactose, maltitol, glycyrrhizin, sodium cyclohexylaminosulfonate, banana flavor, orange flavor, pineapple flavor, mint flavor, fennel, vanillin, lemon flavor, cherry flavor, and rose flavor. The prescription can also optionally contain wetting agent, i.e., aqueous or ethanol solution of different concentrations. The coating material useful in the pharmaceutical preparation of the present invention can be, for example, cellulose and derivatives thereof, acrylic resins, and polymers of ethylene.

The injection preparation includes injectable solution, concentrated solution for injection, or sterile powder for injection. The adjuvant is an additive meeting injection requirements, including pH adjusting agent, isotonic adjustment agent, anti-oxidant, chelating agent, and excipient in the sterile powder for injection. The pH adjusting agent includes hydrochloric acid, lactic acid, methanesulfonic acid, sodium hydroxide, sodium bicarbonate, phosphoric acid and salts thereof, acetic acid and salts thereof, citric acid and salts thereof, and amino acid and salts thereof; the isotonic adjustment agent includes glucose, sodium chloride, glycerol, and sodium sulfate; the excipient includes sorbitol, mannitol, dextran, lactose, sucrose, glucose, hydrolyzed gelatine, and sodium chloride; the anti-oxidant includes 0.01%-0.1% of sodium or potassium pyrosulfite, 0.01-0.5% of sodium sulfite, 0.01%-0.5% of sodium bisulphite, 0.01%-0.5% of sodium thiosulfate, 0.1-0.2% of sodium formaldehydesulfoxylate, 0.05%-0.1% of thiourea, 0.05%-0.2% of ascorbic acid, 0.1%-0.5% of thioglycerol, 0.01-0.2% of glutathione, 0.01%-0.5% of alanine, 0.01%-0.5% of cysteine, 0.01-0.1% of gallic acid and propyl or octyl ester thereof, 0.01-0.1% of tert-butyl p-hydroxyanisole, 0.01%-0.5% of di-tert-butyl p-cresol, 0.01%-0.1% of tocopherol α, β, and γ, 0.01%-0.5% of nordihydroguaiaretic acid, or 0.01%-0.5% of palmityl ascorbate; and the chelating agent includes disodium ethylene diamine tetraacetate, and calcium sodium ethylene diamine tetraacetate.

The compound of the present invention is useful in preparation of detoxification medicament for improving metabolism.

The present invention further provides a liquid chromatography for determining an optical purity of an optically active N-(α-mercaptopropionyl)glycine, including:

1) chromatographic conditions: the chromatography column has a stationary phase of a chiral column with 3,5-dimethylphenyl-carbamate glycopeptides and a mobile phase of n-hexane/ethanol/glacial acetic acid (80-95:5-20:0.01-1.0), with (90:10:0.1) being preferred; the detection wavelength is 200 nm-230 nm, with 210 nm being preferred; and flow rate of the mobile phase is 0.2-3.0 ml/min, with 1.0 ml/min being preferred;

2) formulation of the sample solution: the sample R-(−)-N-(α-mercaptopropionyl)glycine or S-(+)-N-(α-mercaptopropionyl)glycine is formulated into a solution of 0.1-20 mg/ml (preferably 1 mg/ml) with an organic solvent; and the organic solvent is selected from n-propanol, i-propanol, ethanol, i-butanol, and methanol, with ethanol being preferred; and;

3) determination: the solution is injected into a high performance liquid chromatograph (HPLC), and the chromatogram is recorded and analyzed.

### BRIEF DESCRIPTION OF THE DRAWINGS

No drawings.

### DETAILED DESCRIPTION OF THE INVENTION

### Embodiment 1

### Preparation of R-(−)-N-(mercaptopropionyl)glycine

1) 54.3 g (0.5 mol) of R-(+)-2-chloropropionic acid, 60 g (0.504 mol) of thionyl chloride were added to a 100 ml dry reaction flask, and stirred at reflux for 4 h with moisture isolated. After the reaction was completed, excessive thionyl chloride was distilled out for reuse, and then the fraction having a by of 95-105° C. was collected, to get 55 g of R-(+)-2-chloropropionyl chloride as a colorless liquid with a yield of 86.6%.

2) 29.9 g (0.40 mol) of glycine, 21.2 g (0.20 mol) of anhydrous sodium carbonate, and 250 ml of water were added into a 1000 ml reaction flask, and stirred until dissolved. Cooling with an ice-salt bath, 50.6 g (0.40 mol) of R-(+)-2-chloropropionyl chloride was added dropwise with vigorously stirring, and a saturated solution of anhydrous sodium carbonate was added at the same time, to make the reaction solution weak basic. After addition, stirring was continued for additional 3-5 h till the reaction was completed. The reaction solution was acidified to pH=1 with concentrated hydrochloric acid, extracted with ethyl acetate, and then dried over anhydrous magnesium sulfate. The reaction solution was filtered and concentrated under reduced pressure till a crystal was precipitated. Then, the reaction solution was stood still, filtered, and dried, to get 38.6 g of R-(+)-2-chloropropionyl glycine as white small needle crystal. mp: 120-124° C., [α]ₙ²⁰=+23.8° C. (water).

3) 26.5 g (0.11 mol) of sodium sulfide (Na₂S.9H₂O), 3.52 g (0.11 mol) of sublimed sulfur, and 120 ml of water were added to a 250 ml beaker, and heated with stirring until dissolved, to get a red brown solution of sodium disulfide for later use. 16.4 g (0.10 mol) of R-(+)-2-chloropropionyl glycine, and 5.6 g of anhydrous sodium carbonate were added into a 250 ml reaction flask, and then 100 ml of water was added slowly to prevent the generation of bubbles. After cooling to 0-10° C., the sodium disulfide solution was added dropwise, and then reacted for 10-15 h at 5-15° C. After the reaction was completed, the reaction solution was cooled to about 0° C., to which concentrated sulfuric acid was added dropwise, to adjust pH to approximately 1. Then, the reaction solution was filtered, and then 17 g of zinc powder was added in portion to the filtrate with stirring, and reacted for 3 h at normal temperature till the reaction was completed. The reaction solution was filtered, and then the filtrate was extracted with ethyl acetate, washed with saturated sodium chloride solution, and dried over anhydrous magnesium sulfate. The reaction solution was filtered, concentrated under reduced pressure, and stood still, to get a solid. The solid was filtered out and collected, and recrystallized with ethyl acetate, and then dried under vacuum, to get 8.4 g of R-(−)-N-(α-mercaptopropionyl)glycine as white crystalline solid. mp: 102-104° C., [α]ₙ²⁰=−36.5° C. (water), content: 99.3% (titrated with 0.1 mol/L of iodine titration solution), related substance<2% (thin layer chromatography: silica gel G thin plate, chloroform-acetone-glacial acetic acid (9:3:1), developed with iodine vapor).

1HNMR (DMSO-D6) δ ppm: 1.35 (d, 3H); 2.79 (d, 1H); 3.54 (m, 1H); 3.77 (m, 2H); 8.25 (t, 1H); 12.5 (bs, 1H); MS (m/z) 163; element analysis C₅H₉NO₃S (%): C, 36.65; H, 5.66; N, 8.50; S, 19.68.

### Embodiment 2

### Preparation of S-(+)-N-(α-mercaptopropionyl)glycine

1) 54.3 g (0.5 mol) of S-(−)-2-chloropropionic acid, 60 g (0.504 mol) of thionyl chloride were added to a 100 ml dry reaction flask, and stirred at reflux for 4 h with moisture isolated. After the reaction was completed, excessive thionyl chloride was distilled out for reuse, and then the fraction having a by of 95-105° C. was collected, to get 56 g of S-(−)-2-chloropropionyl chloride as a colorless liquid, with a yield of 88.2%.

2) 29.9 g (0.40 mol) of glycine, 21.2 g (0.20 mol) of anhydrous sodium carbonate, and 250 ml of water were added into a 1000 ml reaction flask, and stirred until dissolved. Cooling with an ice-salt bath, 50.6 g (0.40 mol) of S-(−)-2-chloropropionyl chloride was added dropwise with vigorously stirring, and a saturated solution of anhydrous sodium carbonate was added at the same time, to make the reaction solution weak basic. After addition, stirring was continued for additional 3-5 h till the reaction was completed. The reaction solution was acidified to pH=1 with concentrated hydrochloric acid, extracted with ethyl acetate, and then dried over anhydrous magnesium sulfate. The reaction solution was filtered and concentrated under reduced pressure till a crystal was precipitated. Then, the reaction solution was stood still, filtered, and dried, to get 37.8 g of S-(−)-2-chloropropionyl glycine as white small needle crystal. mp: 120-124° C., [α]ₙ²⁰=−24.2° C. (water).

3) 26.5 g (0.11 mol) of sodium sulfide (Na₂S.9H₂O), 3.52 g (0.11 mol) of sublimed sulfur, and 120 ml of water were added to a 250 ml beaker, and heated with stirring until dissolved, to get a red brown solution of sodium disulfide for later use. 16.4 g (0.10 mol) of S-(−)-2-chloropropionyl glycine, and 5.6 g of anhydrous sodium carbonate were added into a 250 ml reaction flask, and then 100 ml of water was added slowly to prevent the generation of bubbles. After cooling to 0-10° C., the sodium disulfide solution was added dropwise, and then reacted for 10-15 h at 5-15° C. After the reaction was completed, the reaction solution was cooled to about 0° C., to which concentrated sulfuric acid was added dropwise, to adjust pH to approximately 1. The reaction solution was filtered, and then 17 g of zinc powder was added in portion to the filtrate with stirring, and reacted for 3 h at normal temperature till the reaction was completed. Then, the reaction solution was filtered, and then the filtrate was extracted with ethyl acetate, washed with saturated sodium chloride solution, and dried over anhydrous magnesium sulfate. The reaction solution was filtered and concentrated under reduced pressure, and stood still, to get a solid. The solid was filtered and collected, and recrystallized with ethyl acetate, and then dried under vacuum, to get 8.2 g of S-(+)-N-(α-mercaptopropionyl)glycine as white crystalline solid. mp: 102-104° C., [α]ₙ²⁰=+37.5° C. (water), content: 99.1% (titrated with 0.1 mol/L of iodine titration solution), related substance<2% (thin layer chromatography: silica gel G thin plate, chloroform-acetone-glacial acetic acid (9:3:1), developed with iodine vapor).

1HNMR (DMSO-D6) δ ppm: 1.40 (d, 3H): 2.80 (d, 1H); 3.60 (m, 1H): 3.81 (m, 2H); 8.28 (t, 1H); 12.8 (bs, 1H); MS (m/z) 163: element analysis C₅H₉NO₃S (%): C, 36.68; H, 5.65; N, 8.53; S, 19.65.

### Embodiment 3

### Preparation of S-(+)-N-(α-mercaptopropionyl)glycine

1) 54.3 g (0.5 mol) of S-(−)-2-chloropropionic acid, 60 g (0.504 mol) of thionyl chloride were added to a 100 ml dry reaction flask, and stirred at reflux for 4 h with moisture isolated. After the reaction was completed, excessive thionyl chloride was distilled out for reuse, and then the fraction having a by of 95-105° C. was collected to get 54.6 g of S-(−)-2-chloropropionyl chloride as a colorless liquid.

2) 29.9 g (0.40 mol) of glycine, 21.2 g (0.20 mol) of anhydrous sodium carbonate, and 250 ml of water were added into a 1000 ml reaction flask, and stirred until dissolved. Cooling with an ice-salt bath, 50.6 g (0.40 mol) of S-(−)-2-chloropropionyl chloride was added dropwise with vigorously stirring, and a saturated solution of anhydrous sodium carbonate was added at the same time, to make the reaction solution weak basic. After addition, stirring was continued for additional 3-5 h till the reaction was completed. The reaction solution was acidified to pH=1 with concentrated hydrochloric acid, extracted with ethyl acetate, and then dried over anhydrous magnesium sulfate. The reaction solution was filtered and concentrated under reduced pressure till a crystal was precipitated. Then, the reaction solution was stood still, filtered, and dried, to get 38 g of S-(−)-2-chloropropionyl glycine as white small needle crystal.

3) 28.8 g (0.12 mol) of sodium sulfide (Na₂S.9H₂O), 3.84 g (0.12 mol) of sublimed sulfur, and 120 ml of ethanol were added to a 250 ml beaker, and heated with stirring until dissolved, to get a red brown solution of sodium disulfide for later use. 18.3 g of (0.11 mol) of S-(−)-2-chloropropionyl glycine, and 6.3 g of anhydrous sodium carbonate were added into a 250 ml reaction flask, and then 100 ml of water was added slowly to prevent the generation of bubbles. After cooling to 0-10° C., the sodium disulfide solution was added dropwise, and then reacted for 12 h at 0-10° C. After the reaction was completed, the temperature was kept constant, and concentrated sulfuric acid was added dropwise, to adjust pH to approximately 1. The reaction solution was filtered, and then 17 g of zinc powder was added in portion to the filtrate with stirring, and reacted for 3 h at normal temperature till the reaction was completed. The reaction solution was filtered, and then the filtrate was extracted with ethyl acetate, washed with saturated sodium chloride solution, and dried over anhydrous magnesium sulfate. The reaction solution was filtered and concentrated under reduced pressure, and stood still to get a solid. The solid was filtered, collected, recrystallized with ethyl acetate, and then dried under vacuum, to get 8.1 g of S-(+)-N-(α-mercaptopropionyl)glycine as white crystalline solid. Content: 99.3% (titrated with 0.1 mol/L of iodine titration solution), related substance<2% (thin layer chromatography: silica gel G thin plate, chloroform-acetone-glacial acetic acid (9:3:1), developed with iodine vapor).

### Embodiment 4

### Determination of Optical Purity of R-(−)-N-(α-mercaptopropionyl)glycine

Instrument: HPLC, SPD-10 Avp UV detector, LC-10AD pump

Mobile phase: n-hexane-ethanol-glacial acetic acid (90:10:0.1)

Flow rate: 1.0 ml/min

Chromatography column: CHIRALPAK AD-H (with 3,5-dimethylphenyl-carbamate glycopeptide as stationary phase)

Column temperature: 25° C.

Detection wavelength: 210 nm

Sample concentration: 0.5 mg/ml

The optical activity of R-(−)-N-(α-mercaptopropionyl)glycine obtained in Embodiment 1 under the conditions above is determined to be 99.3%.

### Embodiment 5

### Determination of Optical Purity of S-(+)-N-(α-mercaptopropionyl)glycine

Instrument: HPLC, SPD-10AvPUV detector, LC-10AD pump

Mobile phase: n-hexane-ethanol-glacial acetic acid (90:10:0.1)

Flow rate: 1.0 ml/min

Chromatography column: CHIRALPAKAD-H (with 3,5-dimethylphenyl-carbamate glycopeptide as stationary phase)

Column temperature: 25° C.

Detection wavelength: 210 nm

Sample concentration: 0.5 mg/ml

The optical activity of S-(+)-N-(α-mercaptopropionyl)glycine obtained in Embodiment 1 under the conditions above is determined to be 99.2%.

### Embodiment 6

### Preparation of R-(−)-N-(α-mercaptopropionyl)glycine Arginine Salt

10 g of R-(−)-N-(α-mercaptopropionyl)glycine obtained in Embodiment 1, 11.2 g of L-arginine, and 60 ml of 95% methanol were added into a reaction flask, stirred, heated to reflux, and reacted for 3 h. After the reaction was completed, the reaction solution was filtered while being hot, and then the filtrate was cooled to room temperature and placed in a freezer to precipitate a crystal by cooling. The crystal was filtered, collected, and dried to obtain 19.2 g of a white crystalline solid with a yield of 92.8%.

### Embodiment 7

### Preparation of S-(+)-N-(α-mercaptopropionyl)glycine Lysine Salt

10 g of S-(+)-N-(α-mercaptopropionyl)glycine obtained in Embodiment 2, 9.4 g of L-lysine, and 60 ml of 95% methanol were added into a reaction flask, stirred, heated to reflux, and reacted for 3 h. After the reaction was completed, the reaction solution was filtered while being hot, and then the filtrate was cooled to room temperature and placed in a freezer to precipitate a crystal by cooling. The crystal was filtered, collected, and dried to obtain 16.8 g of a white crystalline solid with a yield of 88.6%.

### Embodiment 8

### Preparation of R-(−)-N-(α-mercaptopropionyl)glycine sodium

1) 16.3 g (0.10 mol) of R-(−)—N(α-mercaptopropionyl)glycine, and 45 ml of methanol were placed in a reaction flask, to which 0.15 g of dithiothreitol was added at 15° C., and stirred for 15 min, and then 5 g of molecular sieve was added.

2) 4.65 g of NaOH was slowly added into the methanol solution above in 6 portions.

3) After addition, the reactants were reacted for 1 h at 15° C. with stirring, and then for another 1 h at 20-25° C. with stirring. After the reaction was completed, the molecular sieve was filtered off, and then the filtrate was added into 130 mL of acetone, and fully agitated to be homogeneously mixed.

4) The large amount of white precipitate present in the reaction solution was filtered by suction, and then the filter cake was dried at 80° C., to get 12.8 g of anhydrous R-(−)-N-(α-mercaptopropionyl)glycine sodium.

### Embodiment 9

### Preparation of S-(+)-N-(α-mercaptopropionyl)glycine Sodium

1) 16.3 g (0.10 mol) of S-(+)-N-(α-mercaptopropionyl)glycine, and 45 ml of methanol were placed in a reaction flask, to which 0.15 g of dithiothreitol was added at 15° C., and stirred for 15 min, and then 5 g of molecular sieve was added.

2) 4.65 g of NaOH was slowly added into the methanol solution above in 6 portions.

3) After addition, the reactants were reacted for 1 h at 15° C. with stirring, and then for another 1 h at 20-25° C. with stirring. After the reaction was completed, the molecular sieve was filtered off, and then the filtrate was added into 130 mL of acetone, and fully agitated to be homogeneously mixed.

4) The large amount of white precipitate present in the reaction solution was filtered by suction, and then the filter cake was dried at 80° C., to get 13.1 g of anhydrous S-(+)-N-(α-mercaptopropionyl)glycine sodium.

### Embodiment 10

### Preparation of R-(−)-N-(α-mercaptopropionyl)glycine Tablet

Formula Composition:

| | |
|---|---|
| R-(−)-N-(α-mercaptopropionyl)glycine | 100 g |
| Microcrystalline cellulose | 170 g |
| Pregelatinized starch | 60 g |
| 8% starch slurry | suitable amount |
| Sodium carboxymethyl starch | 7 g |
| Magnesium stearate | 3 g |

Preparation process: Taking the preparation of 1000 R-(−)-N-(α-mercaptopropionyl)glycine tablets as example, the raw and auxiliary materials were respectively pulverized and passed through a sieve of 100 mesh for later use; next, formula amounts of R-(−)-N-(α-mercaptopropionyl)glycine, microcrystalline cellulose, and pregelatinized starch were weighted, and homogeneously mixed to prepare a soft material with 8% starch slurry, which was granulated with a sieve of 20 mesh, dried, and then sized with a sieve of 18 mesh; afterwards, formula amounts of sodium carboxymethyl starch and magnesium stearate were added, homogeneously mixed, and then pressed into tablets. The weight of each tablet is about 345 mg.

### Embodiment 11

### Preparation of Film-Coated R-(−)-N-(α-mercaptopropionyl)glycine Tablet

The naked tablet obtained in Embodiment 10 was coated with a formulated 8% gastric soluble OPADRY (OY-C-7000A) solution in ethanol in a high efficient coating pan; and the amount of the coating powder is 2.0-3.0% by weight of the naked tablet.

### Embodiment 12

### Preparation of Enteric Soluble R-(−)-N-(α-mercaptopropionyl)glycine Tablet

The naked tablet obtained in Embodiment 10 was coated with a formulated 8% enteric soluble OPADRY solution in ethanol in a high efficient coating pan; and the amount of the coating powder is 4.0-5.0% by weight of the naked tablet.

### Embodiment 13

### Preparation of Slow Release R-(−)-N-(α-mercaptopropionyl)glycine Tablet

Formula Composition:

| | |
|---|---|
| R-(−)-N-(α-mercaptopropionyl)glycine | 150 g |
| Hydroxypropylmethyl cellulose (K15M) | 150 g |
| Lactose | 50 g |
| 3% povidone solution in 80% ethanol | suitable amount |
| Magnesium stearate | 5 g |

Preparation process: Taking the preparation of 1000 slow release R-(−)-N-(α-mercaptopropionyl)glycine tablets as example, the raw and auxiliary materials were respectively pulverized and passed through a sieve of 100 mesh for later use; next, formula amounts of R-(−)-N-(α-mercaptopropionyl)glycine, hydroxypropylmethyl cellulose, and lactose were weighted, and homogeneously mixed to prepare a soft material with 3% povidone solution in 80% ethanol, which was granulated with a sieve of 20 mesh, dried, and then sized with a sieve of 18 mesh; afterwards, formula amounts of magnesium stearate were added, homogeneously mixed, and then pressed into tablets. The weight of each tablet is about 360 mg.

### Embodiment 14

### Preparation of R-(−)-N-(α-mercaptopropionyl)glycine Dispersible Tablet

Formula Composition:

| | |
|---|---|
| R-(−)-N-(α-mercaptopropionyl)glycine | 100 g |
| Crosslinked polyvinylpyrrolidone | 15 g |
| Microcrystalline cellulose | 180 g |
| Mannitol | 50 g |
| 60% ethanol solution | suitable amount |
| Sodium dodecyl sulfate | 0.2 g |
| Micro-powder silica gel | 5 g |
| Magnesium stearate | 3 g |
| Stevioside | 5 g |

Preparation process: Taking the preparation of 1000 R-(−)-N-(α-mercaptopropionyl)glycine dispersible tablets as example, the raw and auxiliary materials were respectively pulverized and passed through a sieve of 100 mesh for later use; next, formula amounts of R-(−)-N-(α-mercaptopropionyl)glycine, microcrystalline cellulose, and mannitol were weighted, and homogeneously mixed to prepare a soft material with 60% ethanol solution, which was granulated, dried, and then sized; afterwards, formula amounts of crosslinked polyvinylpyrrolidone, sodium dodecyl sulfate, micro-powder silica gel, magnesium stearate, and stevioside were added, homogeneously mixed, and then pressed into tablets. The weight of each tablet is about 360 mg.

### Embodiment 15

### Preparation of R-(−)-(α-mercaptopropionyl)glycine Capsule

Formula Composition:

| | |
|---|---|
| R-(−)-N-(α-mercaptopropionyl)glycine | 150 g |
| Starch | 45 g |
| 5% povidone solution in 60% ethanol | suitable amount |
| Magnesium stearate | 2 g |

Preparation process: Taking the preparation of 1000 R-(−)-N-(α-mercaptopropionyl)glycine capsules as example, the raw and auxiliary materials were respectively pulverized and passed through a sieve of 100 mesh for later use; next, formula amounts of R-(−)-N-(α-mercaptopropionyl)glycine, and starch were weighted, and homogeneously mixed to prepare a soft material with 5% povidone solution in 60% ethanol, which was granulated, dried, and then sized; afterwards, formula amounts of magnesium stearate was added, homogeneously mixed, and then filled into a capsule.

### Embodiment 16

### Preparation of R-(−)-N-(α-mercaptopropionyl)glycine Granule

Formula Composition:

| | |
|---|---|
| R-(−)-N-(α-mercaptopropionyl)glycine | 100 g |
| Mannitol | 350 g |
| Sucrose | 350 g |
| Sodium carboxymethyl cellulose | 50 g |
| Aspartame | 10 g |
| Maltdextrin | 140 g |
| 5% povidone solution in 80% ethanol | suitable amount |
| Flavor | 1 g |

Preparation process: Taking the preparation of 1000 packages of R-(−)-N-(α-mercaptopropionyl)glycine granule as example, the raw and auxiliary materials were respectively pulverized and passed through a sieve of 100 mesh for later use; next, the flavor was added into 5% povidone solution in 80% ethanol, and then formula amounts of R-(−)-N-(α-mercaptopropionyl)glycine, sucrose, mannitol, sodium carboxymethyl cellulose, aspartame, and maltodextrin were weighted to prepare a soft material with 5% povidone solution in 80% ethanol, which was granulated with a sieve of 16 mesh, dried, and then sized with a sieve of 14 mesh; afterwards, the granulates were sieved to remove the fine powder with a sieve of 60 mesh, and packaged. The weight of each package is about 1 g.

### Embodiment 17

### Preparation of Injectable R-(−)-N-(α-mercaptopropionyl)glycine Solution

Formula Composition:

| | | |
|---|---|---|
| R-(−)-N-(α-mercaptopropionyl)glycine | 100 | g |
| Water for injection | made up to 2000 | ml |

Preparation process: Taking the preparation of 1000 ampoules of injectable R-(−)-N-(α-mercaptopropionyl)glycine solution as example, formula amount of R-(−)-N-(α-mercaptopropionyl)glycine was added into 1600 ml of water for injection, and stirred until completely dissolved; then additional water for injection was added to make up to 2000 ml, and uniformly stirred; afterwards, 0.1% of activated carbon for refinement of injection was added, and uniformly stirred; the drug solution was processed with titanium rod to remove active carbon, and then finely filtered with 0.45 μm and 0.22 μm micro membranes in sequence to a satisfactory clarification; then the solution was detected for intermediates, filled into 2 ml ampoules at 2 ml specification under nitrogen flow if eligible, and then sterilized.

### Embodiment 18

### Preparation of R-(−)-N-(α-mercaptopropionyl)glycine Injection

Formula Composition:

| | |
|---|---|
| R-(−)-N-(α-mercaptopropionyl)glycine | 100 g |
| Dextran 40 | 50 g |
| Sodium bisulphate | 1 g |
| Disodium ethylenediamine tetraacetate | 0.2 g |

Preparation process: Taking the preparation of 1000 vials of R-(−)-N-(α-mercaptopropionyl)glycine for injection as example, formula amounts of R-(−)-N-(α-mercaptopropionyl)glycine, dextran 40, sodium bisulphate, and disodium ethylenediamine tetraacetate were added into 1600 ml of water for injection, and stirred until completely dissolved; next, the pH of the solution was adjusted to 1.5-2.5 with 1 mol/L hydrochloric acid, and then additional water for injection was added to make up to 2000 ml, and uniformly stirred; afterwards, 0.1% of activated carbon for refinement of injection was added, and uniformly stirred; the drug solution was processed with titanium rod to remove active carbon, and then finely filtered with 0.45 μm and 0.22 μm micro membranes in sequence and sterilized under aseptic conditions; the drug solution was detected for the presence of intermediates, pH, content, and clarification, and filled into a penicillin bottle at 2 ml specification if eligible, partially stoppered, freeze dried, stoppered and capped, and then light inspected.

### Embodiment 19

### Preparation of S-(+)-N-(α-mercaptopropionyl)glycine Tablet

Formula Composition:

| | |
|---|---|
| S-(+)-N-(α-mercaptopropionyl)glycine | 100 g |
| Microcrystalline cellulose | 170 g |
| Pregelatinized starch | 60 g |
| 8% starch slurry | suitable amount |
| Sodium carboxymethyl starch | 7 g |
| Magnesium stearate | 3 g |

Preparation process: Taking the preparation of 1000 S-(+)-N-(α-mercaptopropionyl)glycine tablets as example, the raw and auxiliary materials were respectively pulverized and passed through a sieve of 100 mesh for later use; next, formula amounts of S-(+)-N-(α-mercaptopropionyl)glycine, microcrystalline cellulose, and pregelatinized starch were weighted, and homogeneously mixed to prepare a soft material with 8% starch slurry, which was granulated with a sieve of 20 mesh, dried, and then sized with a sieve of 18 mesh; afterwards, formula amounts of sodium carboxymethyl starch and magnesium stearate were added, homogeneously mixed, and then pressed into tablets. The weight of each tablet is about 345 mg.

### Embodiment 20

### Preparation of Film-Coated S-(+)-N-(α-mercaptopropionyl)glycine Tablet

The naked tablet obtained in Embodiment 19 was coated with a formulated 8% gastric soluble OPADRY (OY-C-7000A) solution in ethanol in a high efficient coating pan; and the amount of the coating powder used is 2.0-3.0% by weight of the naked tablet.

### Embodiment 21

### Preparation of Enteric Soluble S-(+)-N-(α-mercaptopropionyl)glycine Tablet

The naked tablet obtained in Embodiment 19 was coated with a formulated 8% enteric soluble OPADRY solution in ethanol in a high efficient coating pan; and the amount of the coating powder used is 4.0-5.0% by weight of the naked tablet.

### Embodiment 22

### Preparation of Slow Release S-(+)-N-(α-mercaptopropionyl)glycine Tablet

Formula Composition:

| | |
|---|---|
| S-(+)-N-(α-mercaptopropionyl)glycine | 150 g |
| Hydroxypropylmethyl cellulose (K15M) | 150 g |
| Lactose | 50 g |
| 3% povidone solution in 80% ethanol | suitable amount |
| Magnesium stearate | 5 g |

Preparation process: Taking the preparation of 1000 slow release S-(+)-N-(α-mercaptopropionyl)glycine tablets as example, the raw and auxiliary materials were respectively pulverized and passed through a sieve of 100 mesh for later use; next, formula amounts of S-(+)-N-(α-mercaptopropionyl)glycine, hydroxypropylmethyl cellulose, and lactose were weighted, and homogeneously mixed to prepare a soft material with 3% povidone solution in 80% ethanol, which was granulated with a sieve of 20 mesh, dried, and then sized with a sieve of 18 mesh; afterwards, formula amount of magnesium stearate was added, homogeneously mixed, and then pressed into tablets. The weight of each tablet is about 360 mg.

### Embodiment 23

### Preparation of S-(+)-N-(α-mercaptopropionyl)glycine Dispersible Tablet

Formula Composition:

| | |
|---|---|
| S-(+)-N-(α-mercaptopropionyl)glycine | 100 g |
| Crosslinked polyvinylpyrrolidone | 15 g |
| Microcrystalline cellulose | 80 g |
| Mannitol | 50 g |
| 60% ethanol solution | suitable amount |
| Sodium dodecyl sulfate | 0.2 g |
| Micro-powder silica gel | 5 g |
| Magnesium stearate | 3 g |
| Stevioside | 5 g |

Preparation process: Taking the preparation of 1000 R-(−)-N-(α-mercaptopropionyl)glycine dispersible tablet as example, the raw and auxiliary materials were respectively pulverized and passed through a sieve of 100 mesh for later use; next, formula amounts of S-(+)-N-(mercaptopropionyl)glycine, microcrystalline cellulose, and mannitol were weighted, and homogeneously mixed to prepare a soft material with 60% ethanol solution, which was granulated, dried, and then sized; afterwards, formula amounts of crosslinked polyvinylpyrrolidone, sodium dodecyl sulfate, micro-powder silica gel, magnesium stearate, and stevioside were added, homogeneously mixed, and then pressed into tablets. The weight of each tablet is about 360 mg.

### Embodiment 24

### Preparation of S-(+)-N-(α-mercaptopropionyl)glycine Capsule

Formula Composition:

| | |
|---|---|
| S-(+)-N-(α-mercaptopropionyl)glycine | 150 g |
| Starch | 45 g |
| 5% povidone solution in 60% ethanol | suitable amount |
| Magnesium stearate | 2 g |

Preparation process: Taking the preparation of 1000 S-(+)-N-(α-mercaptopropionyl)glycine capsules as example, the raw and auxiliary materials were respectively pulverized and passed through a sieve of 100 mesh for later use; next, formula amounts of S-(+)-N-(α-mercaptopropionyl)glycine, and starch were weighted, and homogeneously mixed to prepare a soft material with 5% povidone solution in 60% ethanol, which was granulated, dried, and then sized; afterwards, formula amounts of magnesium stearate was added, homogeneously mixed, and then filled into a capsule.

### Embodiment 25

### Preparation of S-(+)-N-(α-mercaptopropionyl)glycine Granule

Formula Composition:

| | |
|---|---|
| S-(+)-N-(α-mercaptopropionyl)glycine | 100 g |
| Mannitol | 350 g |
| Sucrose | 350 g |
| Carboxymethylcellulose sodium | 50 g |
| Aspartame | 10 g |
| Maltdextrin | 140 g |
| 5% povidone solution in 80% ethanol | suitable amount |
| Flavor | 1 g |

Preparation process: Taking the preparation of 1000 packages of S-(+)-N-(α-mercaptopropionyl)glycine granule as example, the raw and auxiliary materials were respectively pulverized and passed through a sieve of 100 mesh for later use; next, the flavor was added into 5% povidone solution in 80% ethanol, and then, formula amounts of S-(+)-N-(α-mercaptopropionyl)glycine, mannitol, sucrose, sodium carboxymethyl cellulose, aspartame, and maltodextrin were weighted to prepare a soft material with 5% povidone solution in 80% ethanol, which was granulated with a sieve of 16 mesh, dried, and then sized with a sieve of 14 mesh; afterwards, it was sieved to remove the fine powder with a sieve of 60 mesh, and packaged. The weight of each package is about 1 g.

### Embodiment 26

### Preparation of Injectable S-(+)-N-(α-mercaptopropionyl)glycine Solution

Formula Composition:

| | | |
|---|---|---|
| S-(+)-N-(α-mercaptopropionyl)glycine | 100 | g |
| Sodium pyrosulfite | 1 | g |
| Disodium ethylene diamine tetraacetate | 0.2 | g |
| Water for injection | made up to 2000 | ml |

Preparation process: Taking the preparation of 1000 ampoules of injectable S-(+)-N-(α-mercaptopropionyl)glycine solution as example, formula amounts of S-(+)-N-(α-mercaptopropionyl)glycine, sodium pyrosulfite and disodium ethylenediamine tetraacetate were added into 1600 ml of water for injection, and stirred until completely dissolved; then additional water for injection was added to make up to 2000 ml, and uniformly stirred; afterwards, 0.1% of activated carbon for refinement of injection was added, and uniformly stirred; the drug solution was processed with titanium rod to remove active carbon, and then finely filtered with 0.45 μm and 0.22 μm micro membranes in sequence to a satisfactory clarification; then the solution was detected for intermediates, filled into 2 ml ampoules at 2 ml specification under nitrogen flow if eligible, and then sterilized.

### Embodiment 27

### Preparation of S-(+)-N-(α-mercaptopropionyl)glycine for Injection

Formula Composition:

| | |
|---|---|
| S-(+)-N-(α-mercaptopropionyl)glycine | 100 g |
| Dextran 40 | 50 g |

Preparation process: Taking the preparation of 1000 vials of S-(+)-N-(α-mercaptopropionyl)glycine for injection as example, formula amounts of S-(+)-N-(α-mercaptopropionyl)glycine, and dextran 40 were added into 1600 ml of water for injection, and stirred until completely dissolved; next, the pH of the solution was adjusted to 1.5-2.5 with 1 mol/L hydrochloric acid or 1 mol/L sodium hydroxide solution, and then additional water for injection was added to make up to 2000 ml, and uniformly stirred; afterwards, 0.1% of activated carbon for refinement of injection was added, and uniformly stirred; the drug solution was processed with titanium rod to remove active carbon, and then finely filtered with 0.45 μm and 0.22 μm micro membranes in sequence and sterilized under aseptic conditions; the solution was detected for the presence of intermediates, pH, content, and clarification, and filled into a penicillin at 2 ml specification if eligible, partially stoppered, freeze dried, stoppered and capped, and then light inspected.

### Embodiment 28

### Effect of Optically Active N-(α-mercaptopropionyl)glycine Administrated by Intravenous Injection on Serum Biochemical Indicators and Liver Indexes of Mice with Liver Injury Induced by Acetamidophenol

The animals were randomly divided into the following 5 groups of 10 animals according to weight: solvent control group, model group (acetamidophenol, 400 mg/kg, ml/kg, intraperitoneal injection), tiopronin group (MPG), R-(−)-N-(α-mercaptopropionyl)glycine group, and S-(+)-N-(α-mercaptopropionyl)glycine group. After fasting for 12 h, except the solvent control group and model group were given saline by intravenous injection, the other test groups were intravenously injected drugs, and then intraperitoneally injected 400/mg/kg of acetamidophenol immediately, to induce acute liver cell injury. The animals were fed with food at 6 hours after modeling, and then fasted for 16 hours; and at 24 hours after modeling, blood were sampled by eye ball removal, centrifuged, and collected for serum. ALT, and AST were determined following the method of the kit. 10% of liver homogenate was prepared, to measure GSH. The liver and spleen were taken at the same time, weighted, and calculated for organcoefficient. The results are shown in Tables 1 and 2.

TABLE 1
Effect of MPG and optical isomer thereof administrated by intravenous injection on
serum biochemical indicators of mice with liver injury induced by acetamidophenol

| | | | | | | Serum |
|---|---|---|---|---|---|---|
| | | Number | Liver | ALT | AST | total |
| | Dosage | of | coefficient | (Karl-Fischer | (Karl-Fischer | protein |
| Group | (mg/kg) | animals | (mg/g) | unit) | unit) | (mg/ml) |
| Solvent control group | — | 12 | 5.405 ± 0.508 | 30.5 ± 18.1 | 30.0 ± 6.5 | 47.5 ± 4.7 |
| Model group | — | 10 | 6.139 ± 0.683 | 5761.8 ± 2761.8 | 1682.8 ± 1122.0 | 42.9 ± 8.1 |
| Tiopronin group | 800 | 8 | 5.804 ± 0.598 | 648.1 ± 412.1 | Not detected | Not detected |
| | | | | | due to | due to |
| | | | | | insufficient | insufficient |
| | | | | | serum | serum |
| S-(+)-N-(α-mercaptopropionyl)glycine | 800 | 12 | 6.201 ± 0.567 | 2880.2 ± 2578.5 | 1201.2 ± 1123.0 | 43.7 ± 5.7 |
| group | | | | | | |
| R-(−)-N-(α-mercaptopropionyl)glycine | 800 | 12 | 5.816 ± 0.613 | 1812.1 ± 1780.1 | 591.3 ± 528.0 | 43.8 ± 7.4 |
| group | | | | | | |

*P < 005,
**P < 0.01 compared with the model group

It can be known from Table 1 that, compared with the solvent control group, the liver coefficient of the mice in the model group is significantly increased, and inhibition of the elevation of serum ALT is apparent in all the dosed groups, in which the function in the R-(−)-N-(α-mercaptopropionyl)glycine group is higher than that in the S—H—N-(α-mercaptopropionyl)glycine group. The ALT value of tiopronin group is lower than that of R-(−)-N-(α-mercaptopropionyl)glycine group, but in the experiment, after modeling, 4 mouse dead in the tiopronin group die, 2 mouse dead in the model group, and no death occurs in the R-(−)-N-(α-mercaptopropionyl)glycine group. Therefore, the protection against liver injury in mice induced by acetamidophenol in R-(−)-N-(α-mercaptopropionyl)glycine group is superior to that in tiopronin group.

TABLE 2
Effect of MPG and optical isomer thereof administrated by intravenous injection
on liver indexes of mice with liver injury induced by acetamidophenol

| | | | Liver | | | MDA |
|---|---|---|---|---|---|---|
| | | Number | total | Liver | | (nmol/g |
| | Dosage | of | protein | albumin | GSH | wet |
| Group | (mg/kg) | animal | (mg/ml) | (mg/ml) | (mo1/L) | weight) |
| Solvent control group | — | 12 | 17.4 ± 3.6 | 10.0 ± 1.9 | 1.20 ± 0.15 | 288.9 ± 84.5 |
| Model group | — | 10 | 13.0 ± 1.5 | 8.3 ± 0.7 | 0.93 ± 0.12 | 452.2 ± 132.8 |
| Tiopronin group | 800 | 8 | 15.7 ± 1.4 | 9.6 ± 1.1 | 0.99 ± 0.16 | 362.4 ± 98.6 |
| S-(+)-N-(α-mercaptopropionyl)glycine | 800 | 12 | 16.2 ± 2.9 | 9.1 ± 2.0 | 0.99 ± 0.16 | 440.0 ± 134.6 |
| group | | | | | | |
| R-(−)-N-(α-mercaptopropionyl)glycine | 800 | 12 | 16.5 ± 1.7 | 9.2 ± 1.3 | 1.11 ± 0.16 | 321.7 ± 72.9 |
| group | | | | | | |

*P < 0.05,
**P < 0.01 compared with the model group

It can be known from Table 2 that, compared with the model group, the content of liver total protein is significantly increased in all the dosed groups, liver GSH content is obviously increased and MDA content is decreased in the R-(−)-N-(α-mercaptopropionyl)glycine group, but no such effects are present in the S—H—N-(α-mercaptopropionyl)glycine group and tiopronin group. The results indicate that R-(−)-N-(α-mercaptopropionyl)glycine can provide better protection against liver injury.

### Embodiment 29

### Protection of N-(α-mercaptopropionyl)glycine and Optical Isomer Thereof Against Liver Injury in Rats Induced by Carbon Tetrachloride

The animals were randomly divided into the following 5 groups of 10 animals according to weight: solvent control group, model group (50% CCl₄, 2 ml/kg, intraperitoneal injection), tiopronin group (MPG), R-(−)-N-(α-mercaptopropionyl)glycine group, and S-(+)-N-(α-mercaptopropionyl)glycine group. Except the solvent control group was given olive oil by intraperitoneal injection, the other test groups were intragastrically administrated drugs for 4 days, once daily, intraperitoneally injected CCl₄ once on the fifth day to induce acute liver cell injury, treated with drugs 30 min before modeling (except for the model control group), and then administrated drugs once 2 hours after modeling. Then, the animals were fasted for 16 h, blood was sampled from retrobulbar venous plexus 24 hours after injecting CCl₄, centrifuged, and collected for serum, and ALT, AST, albumin were determined following the method of the kit. The results are shown in Tables 3, and 4. The left lobe of liver was taken to prepare 10% of liver homogenate and measure GSH and MDA contents. The results are shown in Table 5.

TABLE 3
Effects of MPG and optical isomer thereof on ALT, and AST of rats with
liver injury induced by CCl₄

| | Dosage | ALT (Karl-Fischer | AST (Karl-Fischer |
|---|---|---|---|
| Group | (mg/kg, ig) | unit) | unit) |
| Solvent control group | — | 327.6 ± 11.4 | 117.6 ± 46.5 |
| Model group | — | 1020.1 ± 275.0 | 1448.1 ± 362.5 |
| Tiopronin group | 450 | 748.6 ± 260.2 | 1126.0 ± 159.2 |
| S-(+)-N-(α-mercaptopropionyl)glycine | 450 | 532.8 ± 352.7 | 818.3 ± 347.0 |
| group | | | |
| R-(−)-N-(α-mercaptopropionyl)glycine | 450 | 432.8 ± 252.6 | 718.7 ± 336.2 |
| group | | | |

*P < 0.05,
**P < 0.01 compared with the model group

It can be known from Table 3 that, all the three drugs can significantly decrease the ALT and AST level, in which the decrease in the S-(+)-N-(α-mercaptopropionyl)glycine group and the R-(−)-N-(α-mercaptopropionyl)glycine group is higher than that in the tiopronin group.

TABLE 4
Effects of MPG and optical isomer thereof on serum protein
content of rats with liver injury induced by CCl₄

| | Dosage | Total | | | |
|---|---|---|---|---|---|
| | (mg/kg, | protein | Albumin | Globulin | |
| Group | ig) | (mg/ml) | (mg/ml) | (mg/ml) | A/G |
| Solvent control group | — | 76.2 ± 5.14 | 30.1 ± 2.23 | 46.1 ± 5.13 | 0.660 ± 0.087 |
| Model group | — | 59.1 ± 7.56 | 26.8 ± 2.74 | 32.3 ± 4.99 | 0.837 ± 0.059 |
| Tiopronin group | 450 | 59.8 ± 6.26 | 26.8 ± 1.98 | 32.9 ± 4.79 | 0.825 ± 0.101 |
| S-(+)-N-(α-mercaptopropionyl)glycine | 450 | 64.7 ± 6.40 | 27.5 ± 1.44 | 37.2 ± 5.06 | 0.747 ± 0.073 |
| group | | | | | |
| R-(−)-N-(α-mercaptopropionyl)glycine | 450 | 70.6 ± 3.63 | 30.3 ± 1.20 | 40.3 ± 2.98 | 0.754 ± 0.050 |
| group | | | | | |

**P < 0.01 compared with the model group

It can be known from Table 4 that, compared with the model group, in the R-(−)-N-(α-mercaptopropionyl)glycine group, the serum total protein, albumin and globulin contents are obviously increased, and A/G value is decreased; A/G value is also decreased in the S-(+)-N-(α-mercaptopropionyl)glycine group; but no obvious effect on the indexes are detected in the tiopronin group, which corresponds to the results obtained in the model group.

1-12. (canceled)

13. An optically active N-(α-mercaptopropionyl)glycine, being levo or dextro N-(α-mercaptopropionyl)glycine;
wherein:
the levo N-(α-mercaptopropionyl)glycine is S-(−)-N-(α-mercaptopropionyl)glycine: 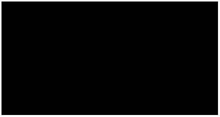
of S-configuration, or a pharmaceutically acceptable salt or ester thereof; and
the dextro N-(α-mercaptopropionyl)glycine is R-(+)-N-(α-mercaptopropionyl)glycine: 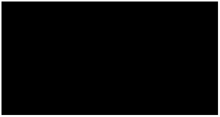
of R-configuration, or a pharmaceutically acceptable salt or ester thereof.

14. The optically active N-(α-mercaptopropionyl)glycine according to claim 13, wherein the salt is an amino acid salt or a metal salt, the amino acid salt has a general structural formula below: 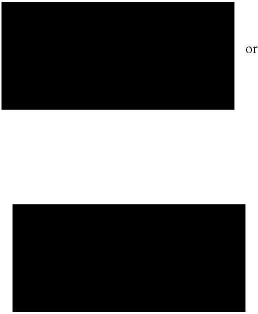
wherein R is an amino acid selected from arginine, lysine, glycine, aspartic acid, alanine, phenylalanine, leucine, isoleucine, ornithine, cystine, cysteine, tyrosine, valine, serine, histidine, threonine, tryptoophan, methionine, methionine, proline, glutamic acid, or hydroxyproline; and
the metal salt has a general structural formula below: 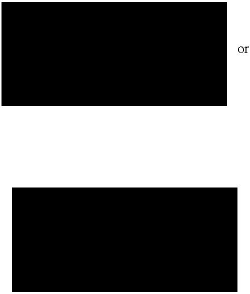
wherein R₀ is potassium, or sodium.

15. The optically active N-(α-mercaptopropionyl)glycine according to claim 13, wherein the ester has a general structural formula below: 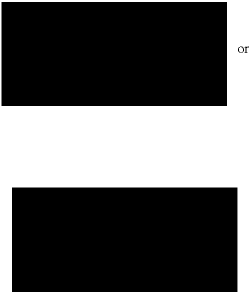
wherein R₁ is a linear C₁-C₅ alkyl group.

16. A method for preparing the optically active N-(α-mercaptopropionyl)glycine according to claim 13, comprising:
reacting R-(+)-2-chloropropionic acid with thionyl chloride to get R-(+)-2-chloropropionyl chloride; next, reacting R-(+)-2-chloropropionyl chloride with glycine under a weak basic condition to get R-(+)-2-chloropropionyl glycine; and then, reacting R-(+)-2-chloropropionyl glycine with sodium disulfide to get S-(−)-N-(α-mercaptopropionyl)glycine;
or comprising:
reacting S-(−)-2-chloropropionic acid with thionyl chloride to get S-(−)-2-chloropropionyl chloride; next, reacting S-(−)-2-chloropropionyl chloride with glycine under a weak basic condition to get S-(−)-2-chloropropionyl glycine; and then, reacting S-(−)-2-chloropropionyl glycine with sodium disulfide to get R-(+)-N-(α-mercaptopropionyl)glycine;
wherein the sodium disulfide is prepared by reacting sodium sulfide with sublimed sulfur.

17. A pharmaceutical preparation comprising the optically active N-(α-mercaptopropionyl)glycine according to claim 13, or a pharmaceutically acceptable salt or ester thereof.

18. The pharmaceutical preparation according to claim 17, wherein the medicament comprising S-(−)-N-(α-mercaptopropionyl)glycine or R-(+)-N-(α-mercaptopropionyl)glycine, or a pharmaceutically acceptable salt or ester thereof as active ingredient, and a pharmaceutically acceptable adjuvant.

19. The pharmaceutical preparation according to claim 17, wherein the preparation is an orally administrated preparation, comprising a general oral preparation, a rapid release oral preparation, or a slow release preparation, and the general oral preparation comprises tablet, capsule, granule, chewable tablet, or effervescent tablet, the rapid release oral preparation is dispersible tablet, or orally disintegrating tablet, and the slow release preparation is slow release tablet or slow release pellet.

20. The pharmaceutical preparation according to claim 17, wherein the preparation is an injection preparation, comprising injectable solution, concentrated solution for injection, or sterile powder for injection.

21. The pharmaceutical preparation according to claim 18, wherein the adjuvant in the oral pharmaceutical preparation comprises a filler, a binder, or a disintegrant, wherein the weight contents of the filler and the disintegrant are 10-60%, and 2-30% respectively; and a glidant, a lubricant, and/or a surfactant optionally, wherein the weight contents of the glidant, the lubricant, and the surfactant are 0.1-5%, 0.1-5%, and 0.005-1% respectively.

22. The pharmaceutical preparation according to claim 19, wherein the adjuvant in the oral pharmaceutical preparation comprises a filler, a binder, or a disintegrant, wherein the weight contents of the filler and the disintegrant are 10-60%, and 2-30% respectively; and a glidant, a lubricant, and/or a surfactant optionally, wherein the weight contents of the glidant, the lubricant, and the surfactant are 0.1-5%, 0.1-5%, and 0.005-1% respectively.

23. The pharmaceutical preparation according to claim 18, wherein the adjuvant in the injection preparation is an additive meeting injection requirements, comprising a pH adjusting agent, an isotonic adjustment agent, an anti-oxidant, a chelating agent, and/or an excipient.

24. The pharmaceutical preparation according to claim 19, wherein the adjuvant in the injection preparation is an additive meeting injection requirements, comprising a pH adjusting agent, an isotonic adjustment agent, an anti-oxidant, a chelating agent, and/or an excipient.

25. A method for treating acute and chronic liver diseases, peripheral blood leukopenia caused by chemoradiation therapy, early senile cataract, or vitreous opacities, wherein the acute and chronic liver diseases include acute and chronic hepatitis, viral hepatitis, alcoholic hepatitis or drug induced hepatitis, heavy metal toxic hepatitis, fatty liver, acute and chronic liver injury, and cirrhosis, comprising orally administering or injecting a pharmaceutical preparation according to claim 17.

26. A liquid chromatography for determining an optical purity of an optically active N-(α-mercaptopropionyl)glycine, comprising:
(1) chromatographic conditions: a chromatography column is a chiral column with 3,5-dimethylphenyl-carbamate glycopeptide as stationary phase and n-hexane/ethanol/glacial acetic acid (80-95:5-20:0.01-1.0) as mobile phase; the detection wavelength is 200 nm-230 nm; and the flow rate of mobile phase is 0.2-3.0 ml/min;
(2) formulation of sample solution: sample S-(−)-N-(α-mercaptopropionyl)glycine or R-(+)-N-(α-mercaptopropionyl)glycine is formulated in an organic solvent to give a solution of 0.1-20 mg/ml; wherein the organic solvent is selected from n-propanol, i-propanol, ethanol, i-butanol, and methanol; and
(3) determination: the solution is injected into a high performance liquid chromatograph (HPLC), the chromatogram is recorded and analyzed.

27. The liquid chromatography according to claim 26, wherein the n-hexane/ethanol/glacial acetic acid ratio is 90:10:0.1.

28. The liquid chromatography according to claim 26, wherein the detection wavelength is 210 nm.

29. The liquid chromatography according to claim 26, wherein the flow rate of the mobile phase is 1.0 ml/min.

30. The liquid chromatography according to claim 26, wherein the organic solvent is ethanol.

31. A method for determining a pharmacokinetic profile of an optically active N-(α-mercaptopropionyl)glycine, comprising:
determining the plasma drug concentration in a test animal with high performance liquid chromatography-mass spectrometry (HPLC-MS), wherein:
a process for treating a plasma sample to formulate an injected solution comprises plasma acidification, extraction, and then derivatization, and is as follows:
1. plasma acidification, wherein the acid used for acidification comprises hydrochloric acid, phosphoric acid, perchloric acid, or acetic acid; and wherein the volume ratio of the acid to the plasma sample is (150 μl-250 μl):(2 ml-4 ml);
2. extraction of the acidified plasma, wherein the organic solvent used for extraction comprises ethyl acetate, chloroform, trichloromethane, diethyl ether, or n-hexane; and
3. derivatization of the extract, wherein the useful derivatization reagent comprises phenyl isothiocyanate and 2,3,4,6-tetra-O-acetyl-β-D-pyranoglucose isothiocyanate (GITC) solution; wherein the derivatization temperature is 15-45° C.; and where the derivatization time is 10-30 min;
chromatographic conditions comprise:
mobile phase A: methanol; mobile phase B: an aqueous solution, containing 0.05-0.20 mmol/L of sodium chloride and 5.0-6.0 mmol/L of formic acid, wherein the ratio of A to B is (40-50:50-60);
mass spectrometric conditions comprise:
ionization mode: electrospray ionization; selective ion detection; curved desolvation line (CDL); temperature: 200° C.-300° C.; heating block temperature: 150° C.-250° C.; CDL voltage: 20V-30V; detection voltage: +1.2 kV-+1.8 kV; flow rate of atomizing gas: 1.2 L/min-1.8 L/min; flow rate of drying gas: 1.5 L/min-2.5 L/min; detected ion: derivative of test drug [M+Na]+(m/z): 575.20; internal standard: derivative of N-isobutanoyl-D-cysteine (NIDC) [M+Na]+(m/z): 603.05; as long as the separation degree of the internal standard peak from the major peak meets the requirements of Chinese Pharmacopoeia.

32. The method according to claim 31, wherein the acid used for acidification comprises hydrochloric acid.
